(19) United States
(12) Patent Application Publication
(10) Pub. No.: US 2024/0293629 A1
(43) Pub. Date: Sep. 5, 2024

(54) VAPORIZER CARTRIDGE AND INHALER COMPRISING SUCH A VAPORIZER CARTRIDGE

(71) Applicant: Körber Technologies GmbH, Hamburg (DE)

(72) Inventor: Thomas Müller, Hamburg (DE)

(21) Appl. No.: 18/571,993

(22) PCT Filed: Jun. 15, 2022
(86) PCT No.: PCT/EP2022/066308

§ 371 Date: Dec. 19, 2023

(30) Foreign Application Priority Data
Jun. 22, 2021 (DE) 102021116108.8

(51) Int. Cl. A61M 11/04

(52) U.S. Cl. CPC A61M 11/042; A61M 2205/123; A61M 2205/3653; A61M 2205/8206

(57) ABSTRACT

A vaporizer cartridge for an inhaler includes a housing having a continuous, barrier-free vent forming a flow channel, and a liquid storage tank. The tank has at least one access opening to the vent. A vaporizer unit arranged in the region of each access opening extends over the entire opening. The vaporizer unit has a wick member and heating member for formation of aerosols from the liquid. The vaporizer unit is liquid-permeable, such that liquid is conveyed, at least initially in a capillary manner, from the tank through the vaporizer unit in the direction of the vent and, in the form of an aerosol, inside the vent in the direction of a mouthpiece. At least one absorption element, arranged in the region of the vent, at least in some regions, is configured for absorption of liquid drops formed inside the vent by condensation. A corresponding inhaler includes the vaporizer cartridge.

### CROSS-REFERENCE TO RELATED APPLICATIONS

This application is the U.S. National Stage of PCT/EP2022/066308 filed on Jun. 15, 2022, which claims priority to German Patent Application 102021116108.8 filed on Jun. 22, 2021, the entire content of both are incorporated herein by reference in their entirety.

### FIELD OF THE INVENTION

The invention relates to a vaporizer cartridge as part of an inhaler, comprising a housing having a continuous, barrier-free vent forming a flow channel, and a storage tank for the storage of liquid, wherein the storage tank has at least one access opening to the vent and in the region of each access opening there is arranged a vaporizer unit which extends over the entire access opening, said vaporizer unit having a wick member and a heating member for the formation of aerosols from the liquid supplied from the storage tank, wherein the vaporizer unit is for that purpose configured so as to be liquid-permeable, such that liquid can be conveyed, at least initially in a capillary manner, from the storage tank through the vaporizer unit in the direction of the vent and, in the form of an aerosol, inside the vent in the direction of a mouthpiece.

The invention relates further to an inhaler, configured and adapted for the inhalation of aerosol enriched with active substances, comprising a cartridge carrier comprising at least an electronic control unit and a power source, and a vaporizer cartridge.

### BACKGROUND OF THE INVENTION

Such vaporizer cartridges and inhalers are used in the stimulants industry, in particular in connection with an electronic cigarette, a so-called e-cigarette, and in the medical field in order to allow liquid stimulants and/or liquid medicinal products to be inhaled. In connection with this invention, the term aerosol also includes vapour or an aerosol/vapour mixture. On consumption, a person usually sucks on a mouthpiece of the inhaler, whereby a suction pressure is formed in the flow channel, which generates an air flow through the flow channel. The air flow can, however, also be generated mechanically, for example by a pump. In the flow channel, a vaporized liquid generated and provided by the vaporizer unit is added as a gas phase to the air flow in order to administer an aerosol or an aerosol/vapour mixture to the consuming person. The liquid is stored at or in the vaporizer cartridge. Various mixtures having various constituents with identical or different vapour densities are used as the liquid. A typical mixture for use in an e-cigarette has glycerol and propylene glycol components, for example, optionally enriched with nicotine and/or virtually any desired flavouring substances. For use in the medical or therapeutic field, for example for the inhalation of asthma preparations, the mixture can correspondingly comprise medicinal constituents and active substances.

The individual parts of the vaporizer cartridge, namely the housing, the storage tank and the vaporizer unit, can be combined in a common component, wherein this component is then a single-use article which is designed for a finite number of inhalation drags by a consuming person and, together with a cartridge carrier in the form of a reusable multi-use article, which comprises at least an electronic control unit and a power source, forms an inhaler. The vaporizer cartridge can, however, also be formed only by the assembly of multiple components, wherein individual components, namely in particular the housing and the vaporizer unit, are arranged in the form of multi-use articles in the cartridge carrier and the storage tank, as a separate component, forms the single-use article. Ultimately, the inhaler can variably be used by replacing the single-use article, which usually contains the liquid.

Accordingly, the single-use article and the multi-use article are detachably connected together. The cartridge carrier, as the multi-use article, usually comprises at least an electronic control unit and a power source. The power source can be, for example, an electrochemical primary battery or a rechargeable electrochemical battery, for example a Li-ion battery, by means of which the heating member is supplied with power via the electrical contacts of the vaporizer unit. The electronic and/or electrical control unit serves to control the vaporizer unit within the vaporizer cartridge. The cartridge carrier can, however, also comprise parts of the vaporizer cartridge. The single-use article can be configured so that it can be plugged onto the multi-use article as a plug-on part or inserted into the multi-use article as an insertion part. Instead of a plug-type connection, screw connections or other quick connections can also be used. Connecting the single-use article and the multi-use article produces a mechanical and electrical coupling to form an operational inhaler.

### SUMMARY OF THE INVENTION

The central component, which ultimately determines the use (e.g. as an e-cigarette or as a medical inhaler), is the storage tank, which is filled with a liquid (also referred to hereinbelow as a fluid), as part of the vaporizer cartridge. By means of the liquid-permeable vaporizer unit, the fluid is guided from the storage tank, as a result of at least initially capillary conveying, through the wick member and the heating member. The voltage generated by the power source, which is applied to the heating member, leads to a current flow in the heating member. As a result of the heating resistance, preferably the ohmic resistance, of the heating member, the current flow leads to heating of the heating member and ultimately to vaporization of the fluid located in the vaporizer unit or guided through the vaporizer unit. The gas or vapour, the aerosol and/or the aerosol/vapour mixture generated in this way escapes from the vaporizer unit in the direction of the flow channel. In the flow channel formed by the vent, the formation of aerosols or aerosol/vapour mixture is purposively assisted by mixing with the air flow within the flow channel. The fluid thus has a predefined path with a predefined flow direction, namely in the form of fluid through the wick member to and through the heating member, and in the form of gas/vapour/mist and/or aerosol from the heating member into the flow channel or vent. In the flow channel or vent, the vaporized fluid is carried along by the air flow when a pressure/vacuum pressure is applied to the flow channel or vent in that, for example, a consuming person sucks on the flow channel via the mouthpiece or a pump blows an air flow through the flow channel or vent.

In order that the fluid does not flow from the storage tank directly into the flow channel or vent, the vaporizer unit completely covers the access from the storage tank to the flow channel or vent. In this context, completely covers means that the liquid is necessarily guided through the vaporizer unit, so that the fluid cannot pass directly from the storage tank into the flow channel or vent but must take the “detour” via the wick member and the heating member. The wick member serves on the one hand for the intermediate storage of fluid, in order to make sufficient fluid available for a few draws on the inhaler in particular when the storage tank is almost empty. The wick member serves on the other hand in particular to transport the fluid from the storage tank in the direction of the flow channel or vent and at the same time acts as a kind of return protection in order to prevent fluid and/or vapour from flowing back in the direction of the storage tank.

In the vaporizer cartridge according to the preamble, the vent is configured so as to be continuous and barrier-free. The vent forms a continuous flow channel. This means that the aerosols or aerosol/vapour mixtures inside the vent or flow channel are able to flow freely in the direction of the mouthpiece. The cavity enclosed by the vent is free of obstacles, so that there is a direct and free channel connection from the region of the vaporizer unit to the mouthpiece. The aerosols or aerosol/vapour mixtures supplied to or formed in the vent flow unhindered in the direction of the mouthpiece. Within the meaning of the invention, barrier-free means, in an ideal case, that the flow channel is of constant cross section and has a smooth surface, so as to permit a laminar flow. However, barrier-free explicitly also includes embodiments in which the cross section of the flow channel, starting from the vaporizer unit to the mouthpiece, is variable or not constant and/or there are provided in the flow channel projections, edges, elevations or the like which, however, extend only partially over the cross section of the flow channel and around which the aerosols or aerosol/vapour mixtures are able to flow. In other words, the barrier-free flow channel allows the aerosols or aerosol/vapour mixtures to flow in the direction of the mouthpiece without having to flow through components or the like situated in the flow channel.

As a result of the cooling of the aerosols or aerosol/vapour mixtures on their path to the mouthpiece, inter alia on the inner wall of the vent surrounding the flow channel, liquid drops form by condensation. Expressed differently, the aerosols or aerosol/vapour mixtures are at least partially converted into liquid again as they are transported from the vaporizer unit to the mouthpiece. As a result of the air draught within the vent or flow channel, there is a risk that the liquid drops will enter the mouth of the person using the inhaler. On the one hand, the liquid drops have an adverse impact on the enjoyment and taste experience, in particular in connection with use as an e-cigarette. Furthermore, the direct intake of nicotine, for example, from the liquid via the oral mucosa can lead to undesirable effects, for example nausea. On the other hand, the liquid drops can have an adverse impact on the intake experience, in particular in connection with a medical inhaler.

Accordingly, the object underlying the invention is to provide a compact vaporizer cartridge of the type according to the preamble which effectively prevents liquid from escaping from the mouthpiece. The object consists further in proposing a corresponding inhaler.

This object is achieved by a vaporizer cartridge of the type mentioned at the beginning in that there is arranged in the region of the vent, at least in some portions, at least one absorption element which is configured and adapted for the absorption of liquid drops formed inside the vent by condensation of the aerosol. By means of the or each absorption element, fluid droplets which form can be absorbed while they are still within the vent, so that they do not even reach the mouthpiece and certainly do not reach the mouth of the inhaling person. In other words, fluid droplets which may form can be retained even before they reach the end of the vent, whereby the enjoyment and taste experience or the intake experience is improved significantly.

Preferably, the or each absorption element is arranged along the portion of the vent that extends from the vaporizer unit to the free end of the mouthpiece, behind the vaporizer unit in the flow direction of the aerosol. The or each absorption element is thus arranged precisely where there is the greatest risk of potential droplet formation, so that effective protection against the undesirable ingestion/inhalation of liquid is provided.

A particularly preferred embodiment is characterized in that the absorption element is formed by the vent itself, said vent being of double-walled form at least in some portions to form an inner vent and an outer vent. On the one hand, a particularly compact form of a vaporizer cartridge having an absorption element is thus formed. On the other hand, the risk of droplet formation, that is to say in particular condensation of the aerosols or aerosol/vapour mixtures on the inner wall of the vent, is reduced by the double-walled form, because the aerosol or aerosol/gas mixture is in effect uncoupled from the liquid stored in the storage tank. The double-walled form in effect acts as insulation with respect to the storage tank. The formation of condensate on the inner wall is thus at least made more difficult. The double-walled form can extend over part of the distance from the vaporizer unit to the mouthpiece. Optionally, the entire distance can also be of double-walled form.

Advantageously, the outer vent adjoins the storage tank and is accordingly configured so as to be impermeable, while the inner vent is configured so as to be perforated at least in some portions in order to produce a connection from the flow channel into the outer vent. The impermeable form of the outer vent with respect to the storage tank, the outer vent preferably forms a wall of the storage tank, describes a liquid-tight boundary, such that liquid is reliably prevented from passing in the direction of the flow channel, with the exception of the region of the vaporizer unit. The perforation at least in some portions, for example by means of very fine pores or holes or the like in the wall of the inner vent, ensures or creates a capillary action, such that the droplets which form on the inner wall of the inner vent in particular by condensation, and also any other type of liquid, move through the openings in the direction of the outer vent, that is to say in particular are discharged from the inner vent. The perforation or any other flow connection from the inner vent surrounding the flow channel into the outer vent can extend over the vent in some portions or completely. The perforation can be of regular or irregular form and can be more pronounced, for example, in the vicinity of the vaporizer unit than in the region of the mouthpiece, or vice versa.

Expediently, the wall of the inner vent and the wall of the outer vent are arranged spaced apart from one another to form an intermediate space. A collecting space for the liquid guided from the inner vent into the outer vent is thus created. The liquid removed from the inner vent can thus be stored in the collecting space and optionally discharged via other channels.

A particularly advantageous further development is characterized in that an additional absorption means, for example in the form of a bonded fabric material, is arranged in the intermediate space. The arrangement of bonded fabric material or any other absorbent material in the intermediate space creates an efficient and effective storage medium for liquid, so that the liquid discharged from the inner vent is retained.

Advantageously, the inner vent, or the wall of the inner vent, is formed by the housing itself, and the outer vent, or the wall of the outer vent, is formed by a tubular portion of the mouthpiece which can be connected to the housing. The mouthpiece can be formed in one piece with the housing. In other embodiments, the mouthpiece is formed separately from the housing and can be connected to the housing by, for example, a plug-type connection, a snap-on system or a quick-acting system. In the mounted state, which corresponds to the operational state, a circumferential intermediate wall of the mouthpiece forms the wall with the storage tank. The outer side of the intermediate wall is in contact with a fastening means, for example a sleeve, or in other embodiments directly with the liquid in the storage tank. The inner side of the intermediate wall faces the intermediate space between the outer vent and the inner vent. The bonded fabric material or the like lies against the inner side of the intermediate wall. Other forms and arrangements of the mouthpiece and/or of the housing are, however, likewise possible. The bonded fabric material or the like is thus sandwiched between the wall of the outer vent, whichever component forms said wall, and the wall of the inner vent. A particularly compact vaporizer cartridge with an effective absorption capacity is thus provided.

In a further advantageous embodiment, the absorption element is arranged directly on the inner side of the vent. In this embodiment, a single-walled configuration is provided, in which a bonded fabric material with high inherent rigidity, for example, is provided as the absorption element. The flow channel is thus surrounded directly by the bonded fabric material or the like, so that the condensate is deposited directly and without an intermediate means on the absorption element and is absorbed thereby, which ensures the effective discharge of the liquid from the flow channel.

Preferably, the absorption element is of sleeve-like form and lies against the inner wall of the vent. The absorption element can thus be mounted easily and in a space-saving manner to form a compact vaporizer cartridge.

The object is also achieved by an inhaler of the type mentioned at the beginning in that the vaporizer cartridge is configured and adapted as disclosed herein.

The advantages resulting therefrom have already been described in connection with the vaporizer cartridge and therefore, in order to avoid repetition, reference is made to the preceding comments.

### BRIEF DESCRIPTION OF THE DRAWINGS

Further expedient and/or advantageous features and further developments relating to the vaporizer cartridge and the inhaler will become apparent from the description. Particularly preferred embodiments of the vaporizer cartridge and of the inhaler will be explained in greater detail with reference to the accompanying drawing, in which:

FIG. 1 shows a schematic representation of a first embodiment of an inhaler according to the invention having a cartridge carrier and a vaporizer cartridge in partial section;

FIG. 2 shows a particularly preferred embodiment of a vaporizer cartridge according to the invention in section, which can be combined with a suitable cartridge carrier to form an inhaler according to the invention; and

FIG. 3 shows a schematic representation of a further embodiment of an inhaler according to the invention having a cartridge carrier and a vaporizer cartridge in partial section.

### DETAILED DESCRIPTION OF THE INVENTION

The vaporizer cartridge and the inhaler shown in the drawing are used for the inhalation of aerosols or aerosol/vapour mixtures, enriched with active substances, for example nicotine, from liquids and accordingly are described in connection with an e-cigarette. The vaporizer cartridge and the inhaler can likewise be used for the inhalation of aerosol or aerosol/vapour mixture, enriched with medicinal active substances, from pharmaceutical products and/or food supplements.

The vaporizer cartridge 10 shown, in particular as part of an inhaler 11, comprises a housing 12 having a continuous, barrier-free vent 14 forming a flow channel 13, and a storage tank 15 for storing liquid 16, wherein the storage tank 15 has at least one access opening 17 to the vent 14 and in the region of each access opening 17 there is arranged a vaporizer unit 18 which extends over the entire access opening 17, said vaporizer unit having a wick member 19 and a heating member 20 for the formation of aerosols (or aerosol/vapour mixtures) from the liquid 16 supplied from the storage tank 15, wherein the vaporizer unit 18 is for that purpose configured so as to be liquid-permeable, such that liquid 16 can be conveyed, at least initially in a capillary manner, from the storage tank 15 through the vaporizer unit 18 in the direction of the vent 14 and, in the form of aerosol (or aerosol/vapour mixture), inside the vent 14 in the direction of a mouthpiece 21. For the sake of simplicity, the term aerosol in connection with the invention also includes aerosol/vapour mixtures throughout. Even when mention is made of vapour or gas, aerosols and aerosol/vapour mixtures are also always meant.

The housing 12 with its vent 14 forms at least one flow channel 13. The form of the housing 12, as well as the profile of each flow channel 13, can be almost as desired. It is important that the entry side Eₛ of each flow channel 13 is open to the surroundings, in order to allow air to be drawn in, for example, and that the exit side Aₛ is open in order to allow a vacuum pressure to be applied, for example, in particular by the suction of a consuming person. In this context, open means that the entry side Eₛ and the exit side Aₛ are air-permeable. Two or more flow channels 13 with corresponding entry sides Eₛ and exit sides Aₛ can also be provided. At least from the vaporizer unit 18 to the mouthpiece 21, each flow channel 13 is not only air-permeable but also completely free of obstacles. Where two or more flow channels 13 are provided, either an absorption element 22 can be associated with each flow channel 13, or a common absorption element 22 is provided, which absorption element is arranged in the region of a common portion, preferably immediately before the exit side Aₛ of the vent 14.

In the region of the access opening 17 between the storage tank 15 and the flow channel 13, the vaporizer unit 18 forms a type of liquid barrier which prevents liquid 16 from flowing out of the storage tank 15 directly and in the form of liquid 16 into the flow channel 13. Regardless of the form and configuration of the storage tank 15, two or more storage tanks 15 can also be provided, and regardless of the form and configuration of the housing 12 and the arrangement/positioning of the storage tank 15 relative to the housing 12, the vaporizer unit 18 ensures that liquid 16 is necessarily guided from the storage tank 15 in the direction of the flow channel 13 and at the latest on leaving the vaporizer unit 18 is delivered in the form of vapour or aerosol or aerosol/vapour mixture into the or each flow channel 13. The mouthpiece 21 can be a separate component or part of the vaporizer cartridge 10.

This vaporizer cartridge 10 is distinguished according to the invention in that there is arranged in the region of the vent 14, at least in some portions, at least one absorption element 22 which is configured and adapted for the absorption of liquid drops formed inside the vent 14 by condensation of the aerosol (or aerosol/vapour mixture). It is accordingly possible that one absorption element 22 or a plurality of absorption elements 22 is associated with, or formed on, the vent 14 over its entire length or over parts of its length.

The vaporizer cartridge 10 according to the invention can, as a single-use article, be a structural unit which contains the components housing 12, storage tank 15 and vaporizer unit 18. The vaporizer cartridge 10 can, however, also be of multi-part form, wherein components of the vaporizer cartridge 10 are divided between the single-use article and the multi-use article, such that, for example, the storage tank 15 is a single-use article which yields the structural unit of the vaporizer cartridge 10 only when combined with a cartridge carrier 23, which can be a multi-use article and can comprise, in addition to an electronic control unit 24 and a power source 25, also components of the vaporizer cartridge 10, such as the housing 12 and the vaporizer unit 18, for example. The vaporizer cartridge 10 is accordingly defined by means of its components, namely the housing 12 having the vent 14 forming the flow channel 13, the storage tank 15 and the vaporizer unit 18, and not by means of the constructive/structural association of the components with the multi-use article or with the single-use article.

The features and further developments described below represent preferred embodiments when considered on their own or in combination with one another. It is explicitly pointed out that features that are combined in the description and/or in the drawing or that are described in a common embodiment can also functionally independently further develop the vaporizer cartridge 10 described above.

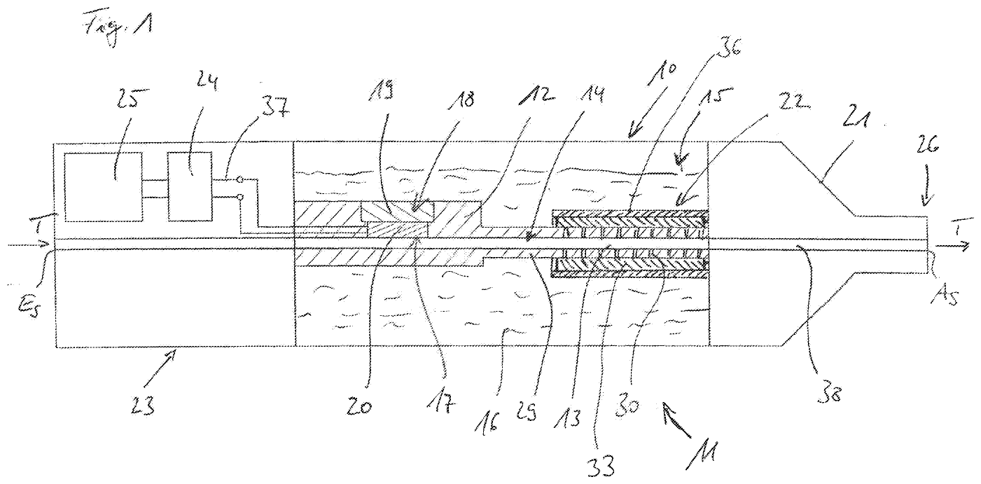

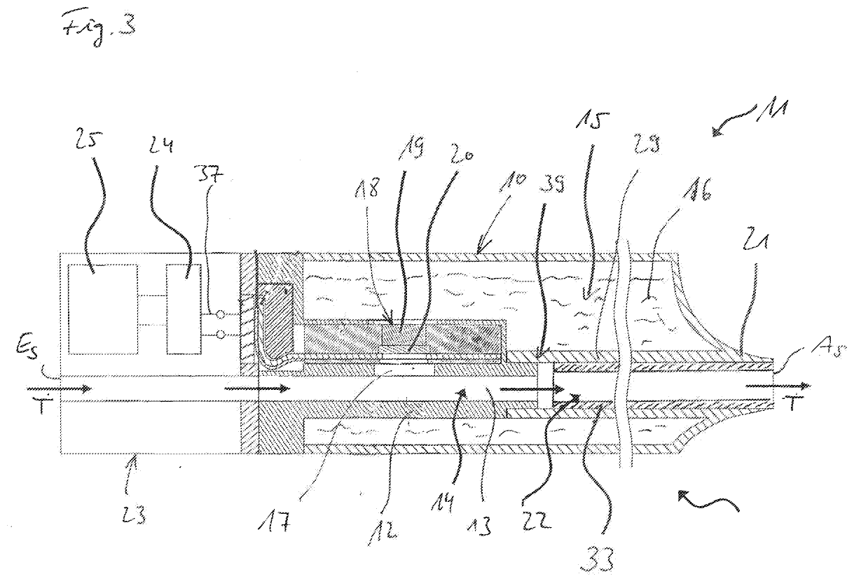

Various embodiments of the vaporizer cartridge 10 according to the invention or of the corresponding inhaler 11 are shown in FIGS. 1 to 3. The or each absorption element 22 is arranged along the portion of the vent 14 that extends from the vaporizer unit 18 to the free end 26 of the mouthpiece 21, behind the vaporizer unit 18 in the flow direction T of the aerosol. The or each absorption element 22 can be in one-part or multi-part form. In other embodiments, the absorption element 22 can also be an integral part of existing components of the vaporizer cartridge 10, in particular of the housing 12. In the embodiment according to FIG. 1, a single absorption element 22 is provided, said absorption element extending along a portion of the vent 14 and being positioned between the vaporizer unit 18 and the mouthpiece 21. The absorption element 22 can also be arranged wholly in the mouthpiece 21 or extend along the entire length from the vaporizer unit 18 to the free end 26 of the mouthpiece 21. A plurality of absorption elements 22 can also be arranged along that distance. FIG. 3 shows a further embodiment having an absorption element 22 that is formed separately from the housing 12 and/or vent 14. The concrete form of the absorption elements 22 in the embodiments according to FIGS. 1 and 3 will be discussed hereinbelow.

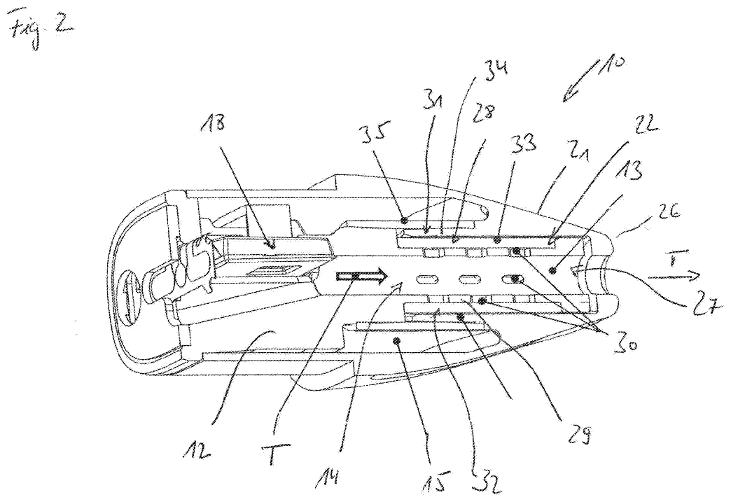

Preferably, the absorption element 22 is formed—at least in part or in some portions—by the vent 14 itself, said vent being of double-walled form at least in some portions to form an inner vent 27 and an outer vent 28. In structurally simple embodiments, the vent 14 formed by the housing 12 is of double-walled form from the beginning to the end, that is to say from the entry side Eₛ to the exit side Aₛ. The double-walled vent 14 can, however, also be formed by a plurality of separate components. A particularly preferred embodiment is shown in FIG. 2. In this embodiment, the inner vent 27 and the outer vent 28 are formed by the configuration of the housing 12 and the mouthpiece 21, on the one hand, and the positioning thereof relative to one another, on the other hand. The outer vent 28 adjoins the storage tank 15 directly or indirectly and is accordingly configured so as to be impermeable. The inner vent 27 is configured so as to be perforated at least in some portions in order to produce a connection from the flow channel 13 into the outer vent 28. The inner vent 27 is formed by a hole/opening in the housing 12. The inner vent 27 thus encloses the flow channel 13 with a wall 29. In the wall 29 of the inner vent 27 there is formed a perforation 30, which can be formed of very fine capillaries, holes or the like. The outer vent 28 is formed by a circumferential, liquid-tight wall 31 of the mouthpiece 21, said wall being completely closed with respect to the storage tank 15. Other components of the vaporizer cartridge 10, optionally supplemented by separate components, can, however, likewise be used to form the inner vent 27 and the outer vent 28.

In the embodiment shown, the wall 29 of the inner vent 27 and the wall 31 of the outer vent 28 are arranged spaced apart from one another to form an intermediate space 32. The intermediate space 32 can be free/empty in order to form, for example, an air cushion having an insulating effect and to act as a storage space for the liquid 16 supplied to the intermediate space 32. In the embodiment shown, an additional absorption means 33, for example in the form of a bonded fabric material, is arranged in the intermediate space 32. The or each absorption means 33 can fill the intermediate space 32 completely or partially. In addition to bonded fabric mats, bonded fabric strips or the like, other materials suitable for the absorption of liquids 16 can also be used and arranged in the intermediate space 32.

As already described, the inner vent 27, or the wall 29 of the inner vent 27, is formed by the housing 12 itself, and the outer vent 28, or the wall 31 of the outer vent 28, is formed by the mouthpiece 21 which can be connected to the housing 12. For this purpose, the mouthpiece 21 has a tubular portion 34 for forming the wall 31. For fastening and positioning the mouthpiece 21 to and on the housing 12 there is provided a fastening means 35, for example in the form of a sleeve, by means of which the tubular portion 34, and thus the mouthpiece 21, can be fixed. Of course, the mouthpiece 21 and the housing 12 can also be connected together in a different way. Furthermore, the configuration of the outer vent 28 can also be implemented differently in terms of construction.

In other embodiments, in which the or each absorption element 22 is not formed by the vent 14 itself, the or each absorption element 22 is associated with the vent from the inside and/or outside. In the embodiment according to FIG. 1, the absorption element 22 comprises a sleeve-like tubular portion 36 and an additional absorption means 33. The absorption element 22 lies against the outside of the vent 14. The mouthpiece 21 is formed separately to the vaporizer cartridge 10, that is to say is an independent component with respect to the housing 12. In the mouthpiece 21 there is formed a hole 38 or the like for prolonging the vent 14, wherein the absorption element 22 ends at the end of the vent 14 formed in the housing 12. For discharging condensed aerosols or aerosol/vapour mixtures, that is to say liquid drops on the wall 29 of the vent 14, the wall 29 of the vent 14 is provided with the perforation 30 in the region in which the absorption element 22 is arranged. The tubular portion 36 shields the additional absorption means 33, which is arranged between the tubular portion 36 and the housing 12, completely and in a sealing manner with respect to the storage tank 15. Outside the sheathing by the tubular element 36, the vent 14 formed by the housing 12 is likewise configured without a perforation 30, that is to say so as to be impermeable with respect to the storage tank 15.

FIG. 3 shows an embodiment in which the absorption element 22 is the absorption means 33. The absorption element 22, or the absorption means 33, is arranged directly on the inner side of the vent 14, wherein the vent 14 is formed by the housing 12 and a tubular portion 39 of the mouthpiece 21. The absorption means 33 can be formed of bonded fabric material, for example. Preferably, the absorption element 22, or the absorption means 33, is of sleeve-like form and lies against the inside of the wall 29 of the vent 14. In order that the absorption means 33 of bonded fabric material is retained and sufficiently fixed in the inside of the vent 14, the bonded fabric material or any other suitable absorption means has sufficient inherent rigidity.

Preferably, the vaporizer cartridge 10 is configured and adapted for mechanical and electrical connection to the cartridge carrier 23 comprising at least the electronic control unit 24 and the power source 25 to form the inhaler 11, wherein the vaporizer unit 18 comprises electrical contacts 37 for electrical contacting with the power source 25. The inhaler 11 can be activated, for example, by an inhaling person, for example as an e-cigarette, or can be activated, for example, by a pump, for example as a medical instrument in the case where the person himself is no longer able to suck or cannot suck sufficiently.

The operating principle of the inhaler 11 according to the invention will be described by way of example with reference to an e-cigarette as an inhaler 11, in particular with reference to FIG. 1. The inhaler 11 is configured and adapted for the inhalation of aerosol or aerosol/vapour mixture enriched with active substances and comprises a cartridge carrier 23 comprising at least the electronic control unit 24 and the power source 25, and a vaporizer cartridge 10, which is distinguished according to the invention in that it is configured and adapted as disclosed herein.

A consuming person sucks, for example, on the mouthpiece 21 of the inhaler 11, which is formed of the cartridge carrier 23 and the vaporizer cartridge 10, wherein a liquid 16 is located in the storage tank 15 of the vaporizer cartridge 10, said liquid containing, for example, glycerol, propylene glycol and optionally further active substances and/or flavouring substances. The suction generates a vacuum pressure in the flow channel 13, which in turn activates the control unit 24 via a sensor (not shown). The control unit 24 controls the heating member 20, which is supplied with power by the power source 25. Liquid 16 from the storage tank 15 is transported by means of the wick member 19, at least initially in a capillary manner, through microchannels from the storage tank 15 in the direction of the heating member 20. The liquid 16 is converted into vapour at or in the heated heating member 20, wherein the heating member 20, by virtue of the liquid- and vapour-permeable structure, transports the liquid 16, or the vapour formed therefrom, in the direction of the flow channel 13 and delivers it thereto. When the vapour enters the flow channel 13, aerosols or an aerosol/vapour mixture form as a result of the air flow and are drawn in and inhaled by the consuming person. Liquid drops which possibly form on the path from the vaporizer unit 18 to the mouthpiece 21, in particular by condensation, are discharged from the inner vent 27, absorbed and optionally stored by the or each absorption element 22.

1-10. (canceled)

11. A vaporizer cartridge as part of an inhaler, comprising:
a housing having a continuous, barrier-free vent forming a flow channel;
a storage tank for the storage of liquid;
wherein the storage tank has at least one access opening to the vent and in a region of each access opening there is arranged a vaporizer unit which extends over the entire access opening;
said vaporizer unit having a wick member and a heating member for the formation of aerosols from the liquid supplied from the storage tank, wherein the vaporizer unit is for that purpose configured so as to be liquid-permeable, such that liquid is conveyed, at least initially in a capillary manner, from the storage tank through the vaporizer unit in a direction of the vent and, in a form of an aerosol, inside the vent in a direction of a mouthpiece;
wherein there is arranged in a region of the vent, at least in some portions, at least one absorption element which is configured and adapted for absorption of liquid drops formed inside the vent by condensation of the aerosol.

12. The vaporizer cartridge according to claim 11, wherein the or each absorption element is arranged along a portion of the vent that extends from the vaporizer unit to a free end of the mouthpiece, behind the vaporizer unit in a flow direction T of the aerosol.

13. The vaporizer cartridge according to claim 11, wherein the absorption element is formed by the vent itself, said vent being of double-walled form at least in some portions to form an inner vent and an outer vent.

14. The vaporizer cartridge according to claim 13, wherein the outer vent adjoins the storage tank and is accordingly configured so as to be impermeable, while the inner vent is configured so as to be perforated at least in some portions in order to produce a connection from the flow channel into the outer vent.

15. The vaporizer cartridge according to claim 13, wherein a wall of the inner vent and a wall of the outer vent are arranged spaced apart from one another to form an intermediate space.

16. The vaporizer cartridge according to claim 15, wherein an additional absorption means is arranged in the intermediate space.

17. The vaporizer cartridge according to claim 16, wherein the additional absorption means is a bonded fabric material.

18. The vaporizer cartridge according to claim 15, wherein the inner vent, or the wall of the inner vent, is formed by the housing itself, and the outer vent, or the wall of the outer vent, is formed by a tubular portion of the mouthpiece which can be connected to the housing.

19. The vaporizer cartridge according to claim 11, wherein the absorption element is arranged directly on an inner side of the vent.

20. The vaporizer cartridge according to claim 19, wherein the absorption element is of sleeve-like form and lies against an inner wall of the vent.

21. An inhaler, configured and adapted for the inhalation of aerosol enriched with active substances, comprising:
a cartridge carrier comprising at least an electronic control unit and a power source; and
a vaporizer cartridge according to claim 1.
